(19) United States
(12) Patent Application Publication
(10) Pub. No.: US 2023/0346724 A1
(43) Pub. Date: Nov. 2, 2023

(54) Methods of Making and Using pH Modulating Compositions in the Reproductive System

(71) Applicant: Crapaud Bio Inc., Wooster, OH (US)

(72) Inventor: J. McLeod Griffiss, Wooster, OH (US)

(73) Assignee: Crapaud Bio Inc., Wooster, OH (US)

(21) Appl. No.: 17/300,191

(22) PCT Filed: Oct. 8, 2019
(86) PCT No.: PCT/US2019/055163

§ 371 Date: Apr. 9, 2021

Related U.S. Application Data
(60) Provisional application No. 62/743,286, filed on Oct. 9, 2018.

(51) Int. Cl. A61K 31/192; A61K 9/00; A61K 47/36; A61K 31/194; A61P 15/02; A61K 33/00; A61K 47/10

(52) U.S. Cl. CPC A61K 31/192; A61K 9/0034; A61K 47/36; A61K 31/194; A61P 15/02; A61K 33/00; A61K 47/10

(57) ABSTRACT

The present invention relates generally to compositions (e.g., gels, suppositories, and the like) and methods for modulating the acidity and/or alkalinity of the fluidic environment of human reproductive organs and systems (e.g., vagina, and cervical fluids/mucus). In particular, the compositions and methods of the present invention are useful for modulating the acidity and/or alkalinity of the fluids within the vagina and that bathe the mucosal surfaces therein. The compositions and methods are useful for promoting and/or sustaining colonization of the vagina by beneficial and/or typically nonpathogenic species of commensal microorganisms (e.g., bacterial species). Additionally, the compositions and methods are useful as adjuvants and/or carriers for introducing one or more species of beneficial commensal microorganisms (e.g., bacterial species) into the vagina such as lactic acid producing bacteria, and in particular, species of Lactobacilli.

### FIELD OF THE INVENTION

The present invention relates generally to compositions (e.g., gels, suppositories, and the like) and methods for modulating the acidity and/or alkalinity of the fluidic environment of human reproductive organs and systems (e.g., vagina, and cervical fluids/mucus). In particular, the compositions and methods of the present invention are useful for modulating the acidity and/or alkalinity of the fluids within the vagina and that bathe the mucosal surfaces therein. The compositions and methods are useful for promoting and/or sustaining colonization of the vagina by beneficial and/or typically nonpathogenic species of commensal microorganisms (e.g., bacterial species). Additionally, the compositions and methods are useful as adjuvants and/or carriers for introducing one or more species of beneficial commensal microorganisms (e.g., bacterial species) into the vagina such as lactic acid producing bacteria, and in particular, species of Lactobacilli.

### BACKGROUND OF THE INVENTION

Bacterial vaginosis (“By”) is a condition principally marked by dysbiosis in the microgranismal flora of the vagina. The clinical syndrome known as bacterial vaginosis has been documented and actively studied for decades. The syndrome was consequently distinguished from classic vaginitis and named vaginosis. Although the precise etiology and pathophysiology of bacterial vaginosis is not fully understood, this syndrome accounts, by some estimates, for about 22% to 50% of reported incidences of vaginitis. The syndrome is classically described according to presentation of various symptoms of mucosal inflammation, including vaginal discharge, itching, and burning, but an absence of leukocytic exudate, redness, and swelling. Bacterial vaginosis is the most common cause of abnormal vaginal discharge among women of reproductive age and the prevalence of bacterial vaginosis is about 30% in women of reproductive age, with estimates ranging from 5% to a high of nearly 70% of women worldwide. (See, Hacer H., et al., Ginekol Pol., 83:744-748 (2012); and Kairys, N. and Garg, M., Bacterial Vaginosis, Stat. Pearls, (2018) https://www.ncbi.nlm.nih.gov/books/NBK459216/).

While bacterial vaginosis is seldom life threatening for the afflicted person, the occurrence of these infections can be fairly common in a population and infection has been linked to a variety of serious health risks. For example, a number of studies purport to show bacterial vaginosis as being linked to increased chances of preterm births with increased probability of serious fetal morbidity and even mortality. Bacterial vaginosis associated complications are thought to include chorioamnionitis, miscarriage, preterm birth, premature rupture of membranes, and postpartum endometritis. Indeed, the incidence of preterm birth alone effects more than 10% of pregnancies and is an expensive and potentially dangerous medical condition for both the women affected by bacterial vaginosis and their babies. (See, Chang H. H., et al., Lancet, 381(9862):223-234 (2013) https://doi.org/10.1016/S0140-6736(12)61856-X). A study published by the Institute of Medicine, reported in the U.S. alone in 2005 that preterm births cost at least $26.2 billion with an average cost of nearly $51,600 per birth. (See, Behrman R. E., and Butler A. S., Preterm birth: Causes, Consequences, and Prevention. Washington, DC: The National Academies Press (2007) https://doi.org/10.17226/11622). Bacterial vaginosis has also been linked to pelvic inflammatory disease (“PID”), a condition in which bacteria infect the uterus and/or fallopian tubes. Pelvic inflammatory disease can cause infertility or damage the fallopian tubes, thus increasing the chances of ectopic pregnancy, a life-threatening condition.

Additionally, bacterial vaginosis has been linked to increased susceptibility to human immunodeficiency virus (HIV) infection and transmission as well as increased susceptibility to other sexual transmitted diseases such as herpes simplex virus (HSV), Chlamydia trachomatis, and Neisseria gonorrhoeae. Taken together, these complications account for a very common syndrome among women of childbearing age.

What is needed in the art are compositions and methods to modulate, increase or decrease, vaginal and intravaginal pH levels.

### SUMMARY OF THE INVENTION

The present invention relates generally to compositions (e.g., gels, suppositories, and the like) and methods for modulating the acidity and/or alkalinity of the fluidic environment of human reproductive organs and systems (e.g., vagina, and cervical fluids/mucus). In particular, the compositions and methods of the present invention are useful for modulating the acidity and/or alkalinity of the fluids within the vagina and that bathe the mucosal surfaces therein. The compositions and methods are useful for promoting and/or sustaining colonization of the vagina by beneficial and/or typically nonpathogenic species of commensal microorganisms (e.g., bacterial species). Additionally, the compositions and methods are useful as adjuvants and/or carriers for introducing one or more species of beneficial commensal microorganisms (e.g., bacterial species) into the vagina such as lactic acid producing bacteria, and in particular, species of Lactobacilli.

The present invention further relates to methods of producing and administering pH modulating compositions. More particularly, the present invention provides methods of producing a biological effect comprising lowering vaginal pH in a subject by providing one or more intervaginal and/or intravaginal administrations (instillations) of the inventive compositions.

A preferred embodiment provides, a method of modulating the pH level in the vagina, said method comprising administering an effective amount of pH modulating composition within the vagina, wherein the composition comprises: 1) about 1 to about 10 percent of a gelling agent selected from the group comprising alginic acid, chitosan, gellan gum, or poloxamer; 2) about 1 to about 10 percent of a mucoadhesive agent selected from the group comprising xanthan gum, hydroxypropyl cellulose, hydroxypropyl methyl cellulose, sodium carboxymethyl cellulose, chitosan, olycarbophil, or a crosslinked polyacrylic acid; 3) about 1 to about 10 percent of an acid component selected from the group comprising lactic acid, citric acid, potassium acid tartrate, benzoic acid, alginic acid, sorbic acid, fumaric acid, ascorbic acid, stearic acid, oleic acid, tartaric acid, edetic acid, or malic acid; and 4) water; further wherein the composition is suitable for application within the vagina; wherein the composition forms a gel on contact with vaginal mucus; further wherein the composition maintains an acidic vaginal pH of less than about 4.5. In some of the embodiments, the acidic vaginal pH is from about 3.5 to about 4.5. Also provided are methods wherein an acidic vaginal pH (e.g., of about 3.5 to about 4.5) is maintained for 1-10 days following administration of pH modulating compositions, and more preferably, wherein the acidic vaginal pH is maintained for 7 days following administration of pH modulating compositions.

The present invention further provides, in certain embodiments, a pH modulating composition wherein the gelling agent comprises alginic acid; wherein the mucoadhesive agent comprises xanthan gum; and further, wherein, the acid component comprises lactic acid and potassium acid tartrate.

The present invention is not limited to formulations comprising fluids, gels, or creams. Indeed, other embodiments are provided in solid or semisolid formulations (e.g., suppositories). It is contemplated that pharmaceutical grade suppository formulations provide convenient metered dosing. Suppository formulations are typically supplied to the recipient in peel-back blister packs containing multiple individual doses.

### Definitions

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The term “bacterial vaginosis” (“By”) and similar terms used herein are to be understood in the broad sense as the alterations of vaginal microflora (e.g., bacterial flora) in a woman, as compared to a baseline, reference, or “normal” vaginal microflora. In other words, the term “bacterial vaginosis” and related terms are not limited to a specific vaginal microflora or any particular symptoms observed in a particular woman or population of women. While bacterial vaginosis can manifest itself through a variety of symptoms, some of which are discussed elsewhere in this document, bacterial vaginosis can also be asymptomatic. In certain situations, bacterial vaginosis may be considered a medical condition or disease that requires treatment, while in other situations bacterial vaginosis may be viewed as a benign but undesirable variation of the vaginal microflora.

Many of the microgranismal (e.g., bacterial) species mentioned herein are normal inhabitants of a human body, and are often thus referred to as “commensal” organisms (e.g., bacteria), particularly when they are not associated with any pathological states or conditions. Some other microorganisms (e.g., bacteria) can be described as “pathological,” particularly if they are typically not found in human body, or found in low numbers, and their presence or increased numbers is associated with a pathological state. It is noted that the same microgranismal (e.g., bacterial) species can be classified as both “commensal” or “pathological,” depending on the accepted classification system, pathology paradigm, bacterial numbers, and other factors. The present invention is therefore not limited to the using commensal, pathological, or any other category of bacterial markers.

As used herein, the term “microflora” refers to microorganisms of a particular habitat or host organism, and vaginal microflora refers to microorganisms of the vaginal region.

As used herein, the term “abnormal vaginal microflora” refers to a microflora state that is associated with certain vaginal conditions or diseases. Abnormal vaginal microflora associated with bacterial vaginosis has a number of distinct characteristics, including replacement of Lactobacillus by fastidious anaerobic bacteria.

As used herein, the following terms, “treatment,” “treating,” “palliating” and “ameliorating,” are used interchangeably as context indicates. These terms refer to an approach for obtaining beneficial or desired results including, but not limited to, therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

As used herein, “lactic acid producing bacteria” are those species of commensal bacteria that produce lactic acid via fermentation including, but not limited to, bacteria from genera Lactobacillus, Leucomostoc, Pediococcus, Streptococcus, Lactococcus, and Enterococcus.

The terms “Lactobacillus,” and “Lactobacilli”, as used herein, refer to commensal Gram-positive, facultative anaerobic or microaerophilic, rod-shaped, non-spore-forming bacteria/bacterium that comprise in part the vaginal flora that ferment carbohydrates by one or more fermentative metabolic pathways (e.g.: 1) obligate homofermentative; 2) facultative heterofermentative; and/or 3) the obligate heterofermentative pathway) to produce lactic acid and other weak acids (e.g., hydrogen peroxide, ethanol, and the like) that acidify the human “vagina” and/or “vaginal cavity.” Lactobacillus species contemplated as being important for maintaining healthy vaginal microbiota include, but are not limited to, L. acidophilus, L. brevis, L. coleohominis, L. crispatus, L. gasseri, L. iners, L. reuteri, L. rhamnosus, and L. vaginalis.

As used herein, the term “pH” refers to the concentration of hydrogen ions in a solution, fluid, or at the surface of relevant mucosal tissues (e.g., vagina). As used herein, the term “healthy pH” and “healthy vaginal pH” refer to a baseline range of the pH values in the vagina of from about pH 3.5 to about 4.5. A healthy vaginal pH can become more alkaline by one or more factors or agents that increase the concentration of ⁻OH ions within the vagina such as, but not limited to, physiological processes (e.g., menus, hormonal fluctuations, and the like); exogenous compounds or chemicals (e.g., semen, sweat, feces, therapeutics, such as, birth control pills, antibiotics, antihistamines, and the like); douching, and the like; microbes and microbial activity, such as dysbiosis in vaginal flora; or introduction of physical devices and other perturbations, such as, catamenials, pessaries, intrauterine devices, intercourse, and the like.

As used herein the term “modulate,” “modulation,” and “modulating,” and grammatical equivalents, refers to the act of changing a measured or presumed pH value to a subsequent measured or presumed value, and more preferably, to: 1) the act of lowering the pH in a fluid (e.g., vaginal mucus and intervaginal fluids) or at a mucosal surface such as the lumen of an orifice (e.g., vagina, cervical tissues, anorectal area, anus, rectum, and the like) to a subsequent relatively more acidic value; or 2) to the act of maintaining the pH of a fluid (e.g., vaginal mucus and intervaginal fluids) or at a mucosal surface such as the lumen of an orifice (e.g., vagina, cervical tissues, anorectal area, anus, rectum, and the like) within a desired range of pH values (e.g., from about pH 3.5 to from about pH 4.5). The act of “modulating” can be relative to a measured, presumed, or desired pH value (e.g., healthy vaginal pH) or to series of one or more pH values as measured, or as presumed, from a subject.

The terms “mucosal” and “mucosa” refer to the mucous membranes comprising one or more layers or surfaces of epithelial cells overlaying a layer of loose connective tissue as these structures line various cavities of the body; in the context of the present invention, and particularly as related to the vagina, urethra, and rectum, the mucosa are contactable and amenable to adhering one or more “mucoadhesive” or “bioadhesive” agents. Accordingly, as used herein, the terms “mucoadhesion” (or “mucoadhesive(s)”), and “bioadhesion” (or “bioadhesive(s)”), as the case implies, refer to the phenomenon, or to the adherent agent, which upon contacting a mucosal tissue adheres thereto usually creating a new interface to the mucus layer. (See, CRC Critical Reviews, Ther. Drug Carrier, 5(1):21 (1988)) generally, “mucoadhesion” can be achieved via physical or chemical processes or both. While the present invention is not limited to any particular mechanism(s) of action, it is contemplated that that the mechanism of mucoadhesion is described in the J. Controlled Release, 2:257 (1982); and J. Controlled Release, 18(249):249 (1992). As used herein, “mucoadhesives” and “bioadhesives” refer to one on more adherent agents that adhere to mucosal tissues described herein.

The term “mucoretentive” (or “retentive”) as used herein refers to a degree of resistance to the normal physiological mechanisms of both longitudinal and circular muscle fiber contractions. Also, “mucoretentive” refers to a composition's degree of resistance to washing and dissolving forces of fluids in the vaginal tract.

In the context of the present invention, the term “mucous” or “mucosal fluid” has its ordinary meaning including, but not limited to, the secretions of any mucosa and may contain mucins as that term is defined in “The Merck Index” 15th ed., 2013; and more preferably, to the mucus (and associated compounds) in the vagina.

As used herein, the term “rheology agent” refers to a substance that modulates the viscosity of a composition and/or formulation, preferably, without modifying other properties of the formulation. In some embodiments, the rheology agent acts as a thickener by increasing the viscosity of the composition. In some embodiments, the rheology agent can include a metal silicate. In some embodiments, the rheology agent is sodium magnesium silicate, a silicate of sodium and magnesium. In some embodiments, sodium magnesium silicate is a synthetic silicate clay, having magnesium and sodium silicate. In some embodiments, a rheology agent is used as a binder and bulking agent in personal care products, in part because of its ability to absorb water. Sodium magnesium silicate is effective in slowing the decomposition of certain formulas, and can prevent premature darkening of compositions and prevent premature development of a foul odor thereby improving composition shelf life. The rheology agent(s) may be used in compositions and formulations in an amount of about 0.1%, 0.25%, 0.5%, 0.75%, 1%, 1.5%, 2%, 2.5%, 3%, 3.5%, 4%, 4.5%, 5%, 6%, 7%, 10%, 15%, or greater w/v %, or in an amount within any two of the aforementioned values or between a range defined by these values.

The term “sample,” as used herein, refers to any sample suitable for testing or assaying according to the methods of the present invention or to routine analytic and/or diagnostic techniques for determining pH and/or the identity of cultured microorganisms. One example of a sample can be referred to as a gynecological sample, such as a vaginal swab obtained according to the procedures accepted in the medical field. However, the term “sample” is not limited to vaginal swabs, but can also be used to describe collected vaginal discharge or mucus samples, tissue samples, cell samples, and/or collected microorganisms, or any portion or subunit thereof, that are suitably obtained, processed, transported and stored using various standard procedures. For examples, the samples can be stored in suitable storage or transportation devices, refrigerated, frozen, desiccated, diluted, cultured, divided, passaged, mixed with various additives, mounted on slides, subjected to common molecular or immunological techniques (e.g., amplification, sequencing, immunoprecipitation, and the like) or physicochemical techniques (e.g., spectroscopy, electrophoresis, chromatography, microscopy, nuclear magnetic resonance, and the like).

As used herein, the term “synergism” refers to at least two substances working together to increase the total effect, the combination is more effective than either substance alone.

A “synergistically effective” therapeutic amount or “synergistically effective” amount of an agent or therapy is an amount which, when combined with an effective or sub-therapeutic amount of another agent or therapy, produces a greater effect than when either of the two agents are used alone. In some embodiments, a synergistically effective therapeutic amount of an agent or therapy produces a greater effect when used in combination than the additive effects of each of the two agents or therapies when used alone. The term “greater effect” encompasses not only a reduction in symptoms of the disorder to be treated, but also an improved side effect profile, improved tolerability, improved patient compliance, improved efficacy, or any other improved clinical outcome.

The terms “co-administration,” “administered in combination with,” and their grammatical equivalents, encompass administration of two or more compositions or agents to a subject so that the compositions or agents and/or their metabolites are present in the subject at the same time. Co-administration includes simultaneous administration in separate compositions, administration at different times in separate compositions, or administration in a composition in which both agents are present. Co-administered agents may be in the same formulation. Co-administered agents may also be in different formulations.

A “therapeutic amount,” as used herein, encompasses the amount of a substance (e.g., the compositions of the present invention) that is sufficient to elicit or promote the desired therapeutic benefit and/or prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof. Specifically, as used herein, a “therapeutic amount” of the present compositions is sufficient to successfully modulate the intravaginal pH in a subject between about pH 3.5 and about pH 4.5 for the desired period of treatment, administration, or effect. A “sub-therapeutic amount” of a substance (e.g., the compositions of the present invention) or therapy using the substance is an amount or application less than the effective amount for that substance or therapy, but when combined with an effective or sub-therapeutic amount of another substance or therapy can produce a result desired by the physician, due to, for example, synergy in the resulting efficacious effects, or reduced side effects.

As used herein, the phrase “pharmacologically effective carrier” refers to any carrier approved for use in humans which facilitates delivery of the acidic compositions of the instant invention without interfering with their therapeutic effect. The carrier preferably is an inert vehicle that exhibits no pharmacologic or therapeutic action.

The term “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine.

In some embodiments, the pharmaceutically acceptable base addition salt is chosen from ammonium, potassium, sodium, calcium, and magnesium salts. “Pharmaceutically acceptable carrier” or “pharmaceutically acceptable excipient” includes any and all polymers, thickening agents, bioadhesive, or mucoadhesives, solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions of the invention is contemplated. Supplementary active ingredients can also be incorporated into the compositions. The phrase “pharmaceutically acceptable” further denotes those substances, compounds, materials, compositions, and/or dosage forms which are, within the scope of sound medical judgment, suitable for use in contact with the tissues (e.g., mucosal tissues) of human beings without excessive toxicity, irritation, allergic response, or other problem or complication commensurate with a reasonable benefit/risk ratio.

In some embodiments, the “purity” of any given agent in a composition may be specifically defined. For instance, certain compositions may include, for example, an agent that is at least 80, 85, 90, 91, 92, 93, 94, 95, 96, 97, 98, 99, or 100% pure, including all decimals in between, as measured, for example and by no means limiting, by suitable analytical techniques.

As used herein, the term “subject” refers to an animal, such as a mammal, for example a human. The compositions and methods described herein can be useful in both human therapeutic, pre-clinical, homeopathic and other complementary and alternative medicine applications. In some embodiments, the subject is a mammal, and in some embodiments, the subject is human.

The term “suppository”, as used herein, refers to a solid, or semi solid, predictably meltable, medical preparation roughly formed in a bullet, conical or teardrop, cylindrical, or ovoid shape that is designed to be inserted into a bodily passage or cavity (e.g., rectum, vagina, or urethra) and left to dissolve. The suppository may be of a homogenous solid composition. Alternatively, the suppository may be a heterogeneous solid composition (e.g., having two or more components as in a first surface layer and a second, or more, inner layer or core compositions). Suppositories can be conventional formulation and production techniques including, but not limited to, metered injection and pouring techniques, hand rolling, compression molding, fusion molding, double casting, and the like.

The traditional suppositories include lipophilic excipients that have a melting point at or below human body temperature. The most widely used fatty acid base in this regard is theobroma oil (i.e., cocoa butter). Theobroma oil is a triglyceride vegetable fat extracted from cocoa beans that is comprised primarily of palmitic acid, steario acid and oleic acid. In other embodiments, naturally occurring, synthetic, or combinations thereof, or triglycerides are employed including, but not limited to, FATTIBASE®, Paddock Laboratories, Dublin, Ireland; WECOBEE®, Stepan Co., Northfield, Illinois; WITEPSOL® H-15, Medisca, Inc., Montreal, Canada; HYDRO-KOTE®, Abitec, Columbus, Ohio; SUPPOCIRE® A, Gattefossé, Saint-Priest, France). Methods of making suppositories with varying melting properties are known in the art.

The term “in vivo” refers to an event that takes place in a subject's body. The term “in vitro” refers to an event that takes places outside of a subject's body. For example, an in vitro assay encompasses any assay run outside of a subject assay. In vitro assays encompass cell-based assays in which cells alive or dead are employed. In vitro assays also encompass cell-free assays in which no intact cells are employed.

As used herein, the term “vagina,” is intended to be synonymous and refer to the external and internal genitalia of the mammalian female in the pudendal region of the body. As used herein, the terms “vaginal cavity,” “within the vagina,” and “vaginal interior,” are intended to refer to the space located between the introitus of the vagina and the cervix including the upper vaginal vault, but not the interlabial space, the floor of vestibule or the externally visible genitalia.

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including,” “includes,” “having,” “has,” and “with,” or variants thereof, are used in either the detailed description and/or the claims, such terms are intended to be inclusive in a manner similar to the term “comprising” unless otherwise noted. In this application, the use of “or” means “and/or” unless stated otherwise.

Recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Unless otherwise defined herein, scientific and technical terms used in connection with the present invention shall have the meanings that are commonly understood by those of ordinary skill in the art. The meaning and scope of the terms should be clear, however, in the event of any latent ambiguity, definitions provided herein take precedent over any dictionary or extrinsic definition.

That the present invention may be more readily understood, select terms are defined below.

### DESCRIPTION OF THE INVENTION

The present invention relates generally to compositions (e.g., gels, suppositories, and the like) and methods for modulating the acidity and/or alkalinity of the fluidic environment of human reproductive organs and systems (e.g., vagina, and cervical fluids/mucus). In particular, the compositions and methods of the present invention are useful for modulating the acidity and/or alkalinity of the fluids within the vagina and that bathe the mucosal surfaces therein. The compositions and methods are useful for promoting and/or sustaining colonization of the vagina by beneficial and/or typically nonpathogenic species of commensal microorganisms (e.g., bacterial species). Additionally, the compositions and methods are useful as adjuvants and/or carriers for introducing one or more species of beneficial commensal microorganisms (e.g., bacterial species) into the vagina such as lactic acid producing bacteria, and in particular, species of Lactobacilli.

The present invention further relates generally to pH modulating compositions that, when placed in a body orifice (e.g., vagina) modulate the pH of the fluids and/or at the luminal surfaces of the tissues comprising the orifice. While the present compositions and methods are not bound to any particular mechanism(s) of action or activity, the formulations of the present invention are contemplated promote and/or sustain the relative normal acidity (e.g., about pH 3.5 to about pH 4.5) of the vagina to provide a permissive environment for expanding or and/or maintaining populations of beneficial commensal bacteria (e.g., Lactobacilli) which preclude or retard establishment and/or expansion of colonies of potentially pathogenic microorganisms that can lead to dysbiosis and ultimately to bacterial vaginosis.

Additionally, the formulations of this invention form a bioadhesive or mucoadhesive layer over the mucosal surfaces in the lumen of the orifice (e.g., vagina, cervical tissue, anorectal area, anus, rectum, and the like), preventing fluctuations and helping to maintain the natural vaginal pH levels of the associated fluids and luminal surfaces of the tissues comprising the orifice.

In certain embodiments, the formulations of this invention are hypertonic thus when placed in the vagina they sequester water from the mucus in the cervix thus gelling and thickening the mucus and associated fluids.

The present formulations are preferably acid-buffering to maintain the normal vaginal milieu and corresponding natural vaginal pH of the environment useful to augment the body's natural defenses against dysbiosis, and in certain embodiments, establishment of various pathogenic microbes in the vagina. The formulations of the present invention may also be used to prevent and/or treat vaginitis and/or bacterial vaginosis.

Compositions and methods are provided for: 1) maintaining normal vaginal pH; and/or 2) altering/modulating (e.g., acidifying) the relative alkalinity in the vaginal from its then existing or transient pH level of greater than about pH 4.5, or greater, to a lower more acidic pH level of from about pH 3.5 to about pH 4.5. The compositions and methods of the present invention can be applied to modulating (e.g., lowering) vaginal pH levels in any number of doses or frequencies of administration/instillations. Accordingly, the present invention specifically contemplates providing a first administration/instillation of a composition at time zero, and a subsequent second, third, fourth, etc., administration/instillation from 1, 2, 3, 4, 5, 6, 7, 8, 9, 10, 11, 12, 13, 14, 15, . . . 30, . . . 60, . . . 90, . . . 120, . . . 365, or more, days respectively, until the next administrations/instillations. Thusly, an administration/instillation schedule can proceed with administrations occurring every 1, 2, 3, 4, 5, 6, 7, 8, 9, 10, 11, 12, 13, 14, 15, . . . 30, . . . 60, . . . 90, . . . 120, . . . 365, or more, days separating doses. In preferred embodiments, a first administration of the composition is followed by a subsequent administration from 3 to 7 days later.

One particular method of the present invention is carried out by administering an effective amount of the compositions of the present invention within the vagina prior to or soon after (e.g., from 1, . . . 5, . . . 15, . . . 30, . . . 60, . . . 90, . . . , 120, . . . 240, . . . 480, . . . 1,440, . . . or more, minutes) heterosexual vaginal intercourse. For purposes of this embodiment, an effective amount is an amount of the composition sufficient to: 1) maintain normal vaginal pH levels during and/or immediately after heterosexual intercourse and ejaculation; 2) to lower the instant alkalinity and transiently higher pH levels in the vagina post ejaculation/insemination; or 3) to form a bioadhesive or mucoadhesive film over vaginal surfaces. Accordingly, the present invention further provides compositions and methods with sufficient buffering capacity to maintain the pH of the vagina in a low or acidic pH condition in the presence of ejaculate (typically from about 1 to about 5.0 ml) of semen having an alkaline pH in the range of about 7.2 to about 7.6. The compositions of the present invention may also be used with conventional birth-control or safe-, safer-sex, devices. For example, the compositions can be used in conjunction with condoms (i.e., via lubricants applied to the interior and/or exterior surfaces), diaphragms, cervix caps, or similar products. The compositions of the present invention could also, for example, be released into the vagina by hand, suppository, or conventional catamenial or syringe techniques. The method of administering or delivering the compositions into the vagina is not critical so long as an effective amount of the composition is delivered into the vagina.

In some embodiments, the compositions of the invention are hypertonic (i.e., having a higher water activity or osmotic pressure relative to the mucus in the cervix in normal, healthy females). Generally, female reproductive tract fluids, including cervical mucus, are generally expected to have an osmolality similar to that of blood plasma (normally in the range of from about 290 to about 320 osmoles/kg). Thus, the osmolality of the compositions of this invention, as measured using conventional osmometer, should be higher than the normal osmolality of blood plasma.

Although the compositions of this invention are mainly intended to be used in situations where the vagina has a normal pH, they can also be used in cases where the microbiological vaginal balance has already been upset (e.g., state of dysbiosis or infection); this buffering capacity may assist in returning the vagina to the desired pH range and a more healthy state. In other words, the compositions of this invention may, if desired, can be used to prevent and/or treat, for example, vaginal infections, including, for example, vaginitis or bacterial vaginosis.

A single dose of the present compositions will normally be in the range of about 1 to about 8 ml of the composition; preferably the single dose is about 3 to about 5 ml. Of course, doses higher or lower than these amounts can be used if desired and the skilled administering physician or healthcare consultant (e.g., nurse, nurse practitioner, pharmacist, and the like) will be able to readily adjust dosing amounts and frequencies to obtain the desired results. In other of these embodiments, the recipient/user will be able to adjust composition dosing amounts and/or frequencies according to their comfort and satisfaction with the results and/or in consideration of vaginal pH measurements from accompanying pH test kits or as provided by healthcare workers.

The compositions of the present invention comprise one or more each of: 1) a gelling agent; 2) a mucoadhesive agent; 3) an acid component; and 4) water. More preferably, the compositions of the present invention comprise one or more of: 1) a gelling agent; 2) a mucoadhesive agent; 3) an acid component; 4) a humectant; 5) a preservative; and 6) water. The compositions can optionally comprise one or more meltable (i.e., at or near physiological temperature) hard fats and other inert vehicles suitable for formulation into suppositories.

In still other embodiments, the compositions generally comprise: 1) from about 1 to about 10 percent of one or more gelling agents; 2) from about 1 to about 10 percent of one or more mucoadhesive agents; 3) from about 1 to about 10 percent of one or more acid components; 4) from 0 to about 2 percent of one or more humectants; 5) from 0 to about 2 percent of one or more preservatives; 6) from 0 to about 10 percent of one or more antimicrobial agents and/or contraceptive agents; and 7) water. More preferably, the compositions of the present invention comprise: 1) about 3 to about 5 percent of one or more gelling agents; 2) about 2.5 to about 6 percent of one or more mucoadhesive agents; 3) about 1 to about 7 percent of one or more acid components; 4) about 6 to about 10 percent of one or more humectants preservatives; 5) about 0.1 to about 1 percent of one or more preservatives; 6) about 0.2 to about 5 percent of one or more antimicrobial or contraceptive agents; and 7) water. Gelling agents suitable for use in the compositions of the present invention are generally stable over a wide pH range, especially over the normal acidic pH values found in the vagina of from pH 3.5 to from about pH 4.5. Suitable gelling agents include, for example, alginic acid, carrageenan, chitosan, gellan gum, hyaluronic acid, methylcellulose, carboxymethylcellulose, hydroxypropylcellulose, poloxamers (e.g., certain PLURONIC® brand polymers, Sigma Aldrich, St. Louis, MO; and certain TWEEN® brand copolymers, Sigma Aldrich, St. Louis, MO), and the like. Preferred gelling agents for use in the compositions of the present invention are at least mildly acidic, moderately acidic, or even strongly acidic.

Alginic acid (also called algin or alginate) is a particularly preferred gelling agent. Alginic acid is a generally linear glycouronan polymer containing a mixture of -(1,4)-D-mannosyuronic acid and -(1,4)-L-gulosyuronic acid residues ((C₆H₈O₆)ₙ). Generally, the molecular weight of the alginic acid is the range of about 20 to about 300,000 g/mole, preferably in the range of about 20,000 to about 250,000 g/mole, and most preferably about 240,000 g/mole. Alginic acid is expected to form insoluble alginates by interacting with monovalent and divalent cations (especially Na⁺, K⁺, and Ca⁺⁺) in seminal plasma. The pKₐ of alginic acid ranges from about 1.5 to about 3.5. It is contemplated that alginic acid, or salts of alginic acid, and other gelling agents that are at least mildly acidic, contribute to the acid buffering activity of the compositions of the present invention in an aqueous solution. Additional forms of alginic acid that find use in certain embodiments of the compositions of the present invention include, but are not limited to, sodium alginate (sodium salt of alginic acid; NaC₆H₇O₆); potassium alginate (potassium salt of alginic acid; KC₆H₇O₆); and calcium alginate (sodium alginate; C₁₂H₁₄CaO₁₂).

Alginates swell in contact with water, thereby assisting in maintaining the desired gel or gel-like consistency of the composition at time of storage, administration, an/or upon administration (e.g., within the vagina). Alginic acid may also contribute to the mucoadhesive nature of the present formulations and, therefore, assist in providing mucoadhesive activity. Because of its high molecular weight, alginic acid will not be absorbed by the body. Thus, the compositions' gelling, mucoadhesive, and acid-buffering properties will be maintained so as long as the compositions (e.g., gel(s), suppositories, and the like) remains in the vagina. Moreover, due to the innate mucoadhesive properties of the preferred compositions, the compositions will normally remain within the vagina from about 12 to about 96 hours (or even longer) if not removed by the woman.

In one particular embodiment, compositions are provided comprising free hydroxy groups on the alginate that are esterified with one or more carboxylic acids (e.g., lactic, citric, tartaric, and the like), rather than a mixture of alginate and free carboxylic acids, as this is contemplated to potentially wash out of the vagina, esterases present in (or introduced into) the vagina are further contemplated to release these acids over time thusly forming a “prodrug version” of the compositions that maintains the desired pH range over an extended period (e.g., from 1 to 96 hours, or longer). In related embodiments, the present invention further provides compositions and methods that interact with one or more extracellular enzymes secreted by vaginal microorganisms (e.g., prokaryotes, such as, bacteria and archaea; and/or eukaryotes, such as, fungi, and in particular, Candida albicans) to modulate vaginal pH. The present invention provides compositions and methods wherein interactions beneficial to producing the desired effect of modulating vaginal pH levels include interactions of the present compositions with one or more extracellular enzymes (e.g., oxidoreductases; hydrolases, such as, esterases; lyases; isomerases; and ligases) from one or more microorganisms. In one such representative embodiment, the present invention provides compositions and methods that interact with extracellular enzymes from C. albicans, and in particular, one or more esterases therefrom.

In other embodiments, the gelling agent comprises one or more forms of carrageenans (e.g., kappa carrageenan, iota carrageenan, and lambda carrageenan) which are sulfated linear polysaccharides. The three forms of carrageenan differ in their degree of progressively greater sulfation, wherein kappa carrageenan has one sulfate group per disaccharide, iota carrageenan has two sulfate groups per disaccharide, and lambda carrageenan has three sulfides groups per disaccharide. In some of these embodiments, the compositions comprising carrageenan comprise the lambda forms and/or iota sulfated forms of carrageenan. In particularly preferred embodiments, the compositions comprise iota carrageenan. All forms of carrageenans are high-molecular-weight polysaccharides made up of repeating galactose units and 3,6 anhydrogalactose (3,6-AG), both sulfated and nonsulfated, wherein the units are joined by alternating α-1,3 and β-1,4 glycosidic linkages. Carrageenans have been used in the food processing, pharmaceutical, and cosmetics industries as lubricants, emulsifiers, and stabilizing and dispersing agents. And as such are recognized by the as GRAS agents and additives. In preferred embodiments comprising one or more sulfated forms of carrageenan, the carrageenan agent is of the so-called refined type where excessive cell wall debris in the product have largely been removed (e.g., 70.00, . . . 80.00, . . . 90.00, . . . 99.99%, or greater, purity).

Mucoadhesive agents suitable for use in certain embodiments of the present invention include, for example, xanthan gum, hydroxypropyl cellulose, hydroxypropyl methyl cellulose, sodium carboxymethyl cellulose, chitosan, polycarbophil, carbopol, and the like. In a preferred embodiment, the mucoadhesive gum is xanthan gum, a high molecular weight polysaccharide gum containing D-glucosyl, D-mannosyl, and D-glucosyluronic acid residues and varying proportions of O-acetyl and pyruvic acid acetal containing polysaccharides. The primary structure is a cellulose backbone with trisaccharide side chains; the repeating unit is a pentasaccharide. Generally the molecular weight is greater than about 10⁶ g/mole. In other embodiments, the preferred mucoadhesive agent is hydroxyethyl cellulose. Preferred mucoadhesive agents for use in the compositions of the present invention are at least mildly acidic, moderately acidic, or even strongly acidic.

Acid components are used in the present composition to promote and/or maintain (e.g., modulate) the pH of the vagina within its normal acidic range (e.g., a pH of less than about 4.5, and more preferably, a pH in the range of about 3.5 to about 4.5). Suitable acid components include, for example, simple organic acid, such as, but not limited to, acetic acid, adipic acid, alginic acid, ascorbic acid, benzoic acid, citric acid, edetic acid, ethylenediaminetetracetic acid, fumaric acid, glacial acetic acid, glutamic acid, hydrochloric acid, lactic acid, malic acid, nitric acid, oleic acid, phosphoric acid, propionic acid, potassium acid tartrate, sorbic acid, stearic acid, succinic acid, sulfuric acid, tartaric acid, and the like, or any combination thereof. The acids may be added as free acids, hydrates, or pharmaceutically acceptable salts. Generally the free acids are preferred. Free acids can be converted to the corresponding salts in situ (i.e., within the vagina). It is generally preferred that several acid components are included in the compositions of the present invention to provide increased buffering and modulating capacity.

Alginic acid, is contemplated to function both as a gelling agent and as an acid component in the present compositions. Since alginic acid is not be absorbed by the body, its acid buffering effect are longer lasting as compared to the other acid components which may be absorbed by the body. In general, preferred acid components for use in the compositions of the present invention are at least mildly acidic, moderately acidic, or even strongly acidic. In still further embodiments, the compositions may include an alkalinizing agent such as, but not limited to, ammonia, ammonium carbonate, diethanolamine, monoethanolamine, potassium hydroxide, potassium phosphate dibasic, sodium bicarbonate, sodium borate, sodium carbonate, sodium hydroxide, sodium lactate, sodium phosphate dibasic, trolamine, or any combination thereof. Alkalinizing agents may optionally be included in the compositions in order to balance the overall acidity of the compositions or as necessary to stabilize one or more of the included agents in the overall composition formulation.

In general, the final formulated pH range of particular embodiments of the compositions of the present invention can range from about pH 3.5 to about pH 8.0, depending on the physiologic stage of the woman and the intended use of the product. In certain embodiments, the pH range of the composition is from about 3.5 to about 4.5 (e.g., for a reproductive aged female), from about 4.5 to about 6.8 (e.g., for a menopausal female), from about 4.5 to about 6.5 (e.g., for a pregnant female), or about 6.8 to about 7.8 (e.g., for a reproductive aged female during ovulatory phase). In certain embodiments, the pH range of a composition may be broad enough to be targeted to female subjects in different life/cycle stages. For example, a composition may have a pH range of about 3.5 to about 4.5 and be used in a reproductive aged, non-ovulating female subject, a menopausal female subject, and a pregnant female subject. Multi-composition kits may be supplied with two or more specific compositions each having a desired pH range as noted above and herein.

In some additional exemplary embodiments, the compositions further include one or more antimicrobial agents and/or a contraceptive agents (e.g., nonoxynol-9, octoxynol-9, benzalkonium chloride, phosphorylated hesperidins, sulfonated hesperidins, polystyrene sulfonates, substituted benzenesulfonic acid formaldehyde co-polymers, and H₂SO₄-modified mandelic acids, povidone iodine, itraconazole, ketoconazole, metronidazole, clotrimazole, fluconazole, teraconazole, miconazole, tinidazole, iconazole, chloramphenicol, nystatin, cyclopiroxolamine, and the like).

The compositions of the present invention may also comprise, and some preferably do comprise, one or more humectants, emollients, emulsifiers, and any combination thereof. Suitable humectants include, for example, sugar alcohol (e.g., glycerin, sorbitol, zylitol, mannitol, and the like), hexylene glycol, butylene glycol, polyethylene glycols, propylene glycol glyceryl triacetate, triacetin, dibutyl sebacate, triethyl citrate, alpha hydroxyl acid (e.g., lactic acid), galactoarabinan, or a combination thereof. Glycerol, which is a preferred humectant, is contemplated to prevent the formation of a dry film on the gel when placed within the vagina. Glycerol may also act as a lubricant. In some embodiments, a preferred humectant is a sugar alcohol. In some other preferred embodiments, the emollient is pharmaceutical grade mineral oil, or a purified non-oxidized plant oil or other plant derived product (e.g., rose, raspberry, cocco, corn, cottonseed, peanut, soybean, sesame, apricot, guar gum, ojoba, olive, shea butter, aloe, and the like), cetostearyl alcohol, glyceryl stearate, acemannan, or a combination thereof. Additional agents suitable for use in certain embodiments of the present invention include, but are not limited to, those described in U.S. Pat. No. 9,642,795.

The compositions of the present invention may optionally comprise, and preferably do comprise, a preservative and/or disinfectant. Suitable preservatives and/or disinfectants include, for example, calcium lactate, hibitane acetate, glacial acetic acid, boric acid, sorbic acid, benzoic acid, sodium benzoate, potassium sorbate, sorbital, benzyl alcohol, tocopherol, phenylethyl alcohol, methylparaben, ethylparaben, butylparaben, propylparaben, propylene glycol, propyl paraben, sodium ethyl paraben, sodium propionate, benyalkonium chloride, benzethonium chloride, phenylmercuric nitrate, chlorhexidine, glycerin, and the like. In other embodiments, the optional preservative is a paraben-free preservative. Parabens are a series of parahydroxybenzoates or esters of parahydroxybenzoic acid and are known to cause cytokine release and potential irritation and have been putatively linked to several types of cancer. Examples of parabens include methylparaben, ethylparaben, propylparaben, butylparaben, heptylparaben, isobutylparaben, isopropylparaben, benzylparaben, and their sodium salts. Exemplary paraben-free preservatives include phenethyl alcohol, caprylyl glycol, phenoxyethanol, a sorbate, potassium sorbate, sodium sorbate, sorbic acid, sodium benzoate, benzoic acid, acemannan, oleuropein, carvacrol, cranberry extract, gluconolactone, green tea extract, Helianthus annuus seed oil, Lactobacillus ferment, Usnea barbata extract, polyaminopropyl biguanide, polyglyceryl-3 palmitate, polyglyceryl-6 caprylate, pomegranate extract, Populus tremuloides bark extract, resveratrol, Rosmarinus officinalis leaf extract, benzyl alcohol, or any combination thereof. In some embodiments, compositions of the present disclosure include a preservative in combination with one or more paraben-free preservative, such as phenethyl alcohol and caprylyl glycol. In particular embodiments, a paraben-free preservative comprises phenethyl alcohol in an amount of 56%-60% and caprylyl glycol in an amount of 40%-44% (e.g., phenethyl alcohol and caprylyl glycol; TRISTAT STABIL®, TRI-K Industries, Inc., Toronto, CA). A preferred optional preservative is benzoic acid. As discussed above, benzoic acid may also contribute to the buffering capacity of the gel as might other acidic agents and components of the compositions. In another preferred embodiment, the optional preservative is sorbic acid.

The compositions of this invention preferably contain alginic acid as the gelling agent; xanthan gum and/or hydroxycellulose as the mucoadhesive agent; an acid component selected from the group consisting of lactic acid, citric acid, benzoic acid, potassium acid tartrate; glycerol as the humectant; benzoic acid as the preservative; and water. More preferably, the compositions of this invention contain xanthan gum, alginic acid, lactic acid, citric acid, benzoic acid, potassium bitartrate, glycerol, and water. If additional antimicrobials and/or contraceptives are to be included, the compositions of the invention more preferably contain xanthan gum, alginic acid, lactic acid, citric acid, benzoic acid, potassium bitartrate, glycerol, water, and a antimicrobial and/or a contraceptive agent selected from the group consisting of nonoxynol-9, octoxynol-9, benzalkonium chloride, phosphorylated hesperidins, sulfonated hesperidins, polystyrene sulfonates, substituted benzenesulfonic acid formaldehyde co-polymers, and H₂ SO₄-modified mandelic acids, povidone iodine, itraconazole, ketoconazole, metronidazole, clotrimazole, fluconazole, teraconazole, miconazole, tinidazole, iconazole, chloramphenicol, nystatin, and cyclopiroxolamine.

Suitable antimicrobial and contraceptive agents include, for example, nonoxynol-9, octoxynol-9, benzalkonium chloride, phosphorylated hesperidins, sulfonated hesperidins, polystyrene sulfonates, substituted benzenesulfonic acid formaldehyde co-polymers, H₂SO₄-modified mandelic acids, povidone iodine, itraconazole, ketoconazole, metronidazole, clotrimazole, fluconazole, teraconazole, miconazole, tinidazole, iconazole, chloramphenicol, nystatin, cyclopiroxolamine, and the like. Generally these antimicrobial and contraceptive agents, if used, are included in an amount of less than about 12 percent, and preferably at a level of about 2 to about 6 percent. Nonoxynol-9, a well-known and commercially available contraceptive agent, may cause vaginal irritation in some women; in those cases it may be preferred to lower the concentration, or even eliminate, nonoxynol-9. Suitable phosphorylated hesperidins and sulfonated hesperidins are described in U.S. Pat. No. 5,925,621. Suitable H₂SO₄-modified mandelic acids are described in U.S. Pat. No. 5,932,619. Suitable substituted acid formaldehyde co-polymers are described in U.S. Pat. No. 6,028,115. Especially preferred co-polymers include, but are not limited to, branched poly(methyl ether)hydroquinone sulfonates and derivatives thereof. Suitable polystyrene sulfonates are described in U.S. application Ser. No. 09/252,417. Generally, acid-stable, noncytotoxic agents such as H₂SO₄-modified mandelic acids and branched poly(methyl ether)hydroquinone sulfonates and derivatives thereof, are preferred.

In some embodiments, more particularly, the compositions and formulations are administered in combination with a one or more additional vaginitis therapy or therapies, including antimicrobial agents, anti-inflammatory agents, or antihistamines. As used herein, antimicrobial agents include antibiotics or antifungals. In some embodiments, antibiotics includes aminoglycoside derivative like amikacin, gentamicin, kanamycin, neomycin, netilmicin, tobramycin, paromomycin; an ansamycin derivative like geldanamycin, herbimycin; a carbacephem derivative like loracarbef; a carbapenem derivative like ertapenem, doripenem, imipenem, meropenem; a cephalosporin derivative like cefadroxil, cefazolin, cefalotin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftobiprole; a glycopeptide derivative like teicoplanin, vancomycin, telavancin; a lincosamides like clindamycin, lincomycin; a lipopeptide derivative like daptomycin; a macrolide derivative like azithromycin, clarithromycin, dirithromycin, erythromycin, roxithromycin, troleandomycin; telithreomycin, spectinomycin; a monobactam derivative like aztreonam; a nitrofuran derivative like furazolidone, nitrofurantoin; a penicillin derivative like amoxicillin, ampicillin, azlocillin, carbinicillin, cloxacillin, dicloxacillin, flucloxacillin, mezlocillin, methicillin, nafcillin, oxacillin, penicillin G, penicillin V, piperacillin, temocillin, ticarcillin; a penicillin combination like amoxicillin/clavulanate, ampicillin/sulbactam, piperacillin/tazobactam, ticarcillin/clavulanate; a polypeptide derivative like bacitracin, colistin, polymyxin B; a quinolone derivative like ciprofloxacin, enoxacin, gatifloxacin, levofloxacin, lomefloxacin, moxifloxacin, nalidixic acid, norfloxacin, ofloxacin, trovafloxacin, grepafloxacin, sparfloxacin, temafloxacin; a sulfonamide derivative like mafenide, sulfonamidochrysoidine, sulfacetamide, sulfadiazine, silver sulfadiazine, sulfamethoxazole, sulfanilimide, sulfasalazine, sulfisoxazole, trimethoprim, trimethoprim/sulfamethoxazole; a tetracycline derivative like demeclocycline, doxycycline, minocycline, oxytetracycline, tetracycline; a derivative against mycobacteria like clofazimine, dapsone, capreomycin, cycloserine, ethambutol, ethioamide, isoniazid, pyrazinamide, rifampin, rifampicin, rifabutin, rifapentine, streptomycin; or arsphenamine, chloramphenicol, fosfomycin, fusidic acid, linezolid, metronidazole, mupirocin, platensimycin, quinupristin/dalfopristin, rifaximin, thiampheniol, tigecycline, amphotericins, novobiocins, polymixins, gramicidins, framycetin, ribostamycin, arbekacin, bekanamycin (kanamycin B), dibekacin, hygromycin B, sisomicin, isepamicin, verdamicin, astromicin, neamine, ribostamycin, lividomycin, or tinidazole or any salts or variants thereof. In some embodiments, antibiotics include those that are typically used for the treatment of bacterial vaginosis, including metronidazole or clindamycin. In some embodiments, antifungals include azole, naftifine, ciclopirox, or terbinafine. In certain of these particular embodiments, the antimicrobial agent is administered topically, or deposited into the vagina, or orally as typically indicated for particular agent.

The compositions of this invention are prepared using conventional gel preparation techniques. It is important, however, to ensure that the acid components are fully solubilized in the final product and that the entrapment of air in the gel is avoided or at least kept to a minimum. To reduce the entrapment of air in the gel, it is generally preferred that the less hydrophilic agents (e.g., alginic acid) are added in small increments. Alternatively, the compositions of this invention can also be prepared in readily dispensable solid forms (e.g., powders, tablets, and the like) which can be converted to the desired gel consistency by action of aqueous based fluids external to or within the vagina when desired. As those skilled in the art will realize, the methods for preparing the compositions of this invention can be modified for batch, semi-continuous, or continuous operation so long as the resulting compositions have the desired and beneficial properties described herein.

In further embodiments, compositions of the present invention optionally further comprise an osmolality adjuster or osmolality adjusting agent. Osmolality is a measure of minimum pressure which needs to be applied to a solution to prevent the inward flow of water across a semipermeable membrane and is expressed in milliosmoles per kilogram of solvent (mOsmol/kg). The osmotic pressure of a solution depends on the number of particles in solution. Osmolality of a solution may be measured by measuring freezing point depression or vapor pressure lowering. In certain embodiments, the osmolality range of the compositions disclosed herein is about 200 mOsm/kg to about 600 mOsm/kg, about 240 mOsm/kg to about 400 mOsm/kg, or about 260 mOsm/kg to about 380 mOsm/kg.

In certain embodiments, compositions disclosed herein further comprise a surfactant. In some embodiments, the surfactant is cetyl hydroxyethylcellulose, hydrophobically modified hydroxyethyl cellulose, poloxamer, polyoxyethylene glycol alkyl ether, polyoxypropylene glycol alkyl ether, glucoside alkyl ether, polyoxyethylene glycol alkylphenol ether, glycerol alkyl ester, polysorbate, cocamide monoethaolamine, cocamide diethanolamine, dodecyldimethylamine oxide, or any combination thereof. The surfactant may be present at a concentration of about 0.01% to about 20%, about 0.01% to about 15%, about 0.01% to about 10%, about 0.01% to about 10%, about 0.01% to about 5%, about 0.01% to about 2.5%, about 0.01% to about 1%, about 0.01% to about 0.5%, about 0.01% to about 0.1%, about 0.1% to about 20%, about 0.1% to about 10%, about 0.1% to about 5%, about 0.1% to about 2.5%, 0.1% to about 1%, or about 0.1% to about 0.7%, about 1% to about 20%, about 1% to about 10%, about 1% to about 15%, about 1% to about 10%, about 1% to about 5%, or about 1% to about 2.5% by weight.

In still other embodiments, the compositions are formulated and optimized for use with one or more vaginal prebiotic and/or probiotic compositions to introduce or potentially activate colonies of beneficial commensal bacteria in the vagina and reproductive organs. Exemplary prebiotic and/or probiotic compositions that find use with one or more embodiments of the present invention include, but are not limited to, those described in U.S. Pat. No. 7,312,067; U.S. Publication 20040253217; U.S. Publication 20080057046; U.S. Publication 20180161385; and U.S. Publication 20180228850. Accordingly, in specific embodiments, the compositions disclosed herein further comprise direct additional factors contemplated to help maintain, improve, or enhance vaginal flora (vaginal microbiota) (e.g., Lactobacilli) function and health, such as amylase, glycogen, D-lactic acid, L-lactic acid, or a combination thereof.

In certain embodiments, the compositions of the present invention further comprise at least one prebiotic. A prebiotic refers to non-digestible plant fiber that stimulates the growth or activity of one or more species of vaginal flora. Examples of prebiotics that can be used include acemannan and galactoarabinan. (See, Al-Ghazzewi, F. H., and Tester R. F., J. Appl. Microbiol., 121(1):18-27 ((2016); and Gullón, P., et al., Food Funct., 6:525-531 (2015)). In certain other embodiments, the compositions disclosed herein further comprise at least one vaginal probiotic bacterial species (e.g., belonging to the genus Lactobacillus) or composition. As used herein, “vaginal probiotic bacteria” refer to live bacteria, which when administered in adequate amounts to the vagina confer a health benefit to the host. In certain embodiments, the probiotic bacterial species or strain is one having the ability to colonize the human vagina. The adhesion of Lactobacilli to the uroepithelium varies among species and strains, as shown by in vitro studies (Reid, G., et al., J. Urol. 138:330-335 (1987)) and may be mediated by glycoprotein and carbohydrate adhesins binding to glycolipid receptors. (See, Boris, S., et al., Infection and Immunity, 66:1985-1989 (1998)). In some embodiments, a vaginal probiotic species is a species that is part of the healthy vaginal flora. In specific embodiments, a vaginal probiotic species is selected from Lactobacillus fermentum, Lactobacillus acidophilus, Lactobacillus jensenii, Lactobacillus gassed, Lactobacillus iners, Lactobacillus crispatus, Lactobacillus plantarum, Lactobacillus fermentum, Lactobacillus brevis, Lactobacillus casei, Lactobacillus delbrueckii, Lactobacillus vaginalis, Lactobacillus salivarius, or any combination thereof. In some embodiments, a vaginal probiotic bacterial species or strain is encapsulated or coated. Compositions containing a vaginal probiotic species preferably include a paraben-free preservative.

A vaginal probiotic bacterial species or strain may be administered in an amount of about 10⁴ to about 10¹² colony forming units (CFU), or about 10⁵ to about 10¹¹ CFU, or about 10⁵ to about 10¹⁰ CFU, or about 10⁷ to about 10⁹ CFU per dose/administration. In some embodiments, a vaginal probiotic bacterial species or strain may be administered in an amount of about 4 million to about 400 million colony forming units (CFU) per dose/administration. A vaginal probiotic bacterial species or strain may be included in the compositions disclosed herein related to specific embodiments for treatment and/or prophylaxis of bacterial vaginosis, viral vaginosis, yeast vaginitis, infections in the vagina, sexually transmitted diseases, such as HIV and chlamydia infection, infections endangering the fetus in pregnant women, preterm labor and urinary tract infection as well as general modulation of vaginal acidity and maintenance of normal vaginal pH taking into account physiological status of the recipient.

If desired, flavoring agents, sweetening agents (e.g., aspartame, dextrose, maltose, mannitol, saccharine, xylitol, and the like), fragrances, and colorants can be incorporated into the composition so long as they do not interfere with the utility of the composition. Indeed, incorporation of such flavoring agents, fragrances, and colorants into the compositions of this invention may provide further protection by increasing the probability that the composition will be used during sexual activity. In preferred embodiments, incorporating one or more of these additional entities, any flavoring agents, fragrances, and colorants used are selected from those of their class generally recognized as safe for human use and of a suitable high pharmaceutical grade.

Organisms that Likely Cause Bacterial Vaginosis

Current theories of bacterial vaginosis explain the condition as the replacement of the normal, homogeneous, vaginal microflora (i.e., Lactobacilli) with a heterogeneous mix of anaerobic and microaerophilic organisms. Accordingly, while not being bound to any particular mechanism(s), the present invention contemplates bacterial vaginosis as being caused by dysbiosis in the species of naturally occurring bacteria present in healthy vaginal flora wherein the normally predominant species of Lactobacilli are markedly reduced while the abundance of potentially deleterious and/or opportunistic bacterial species are markedly increased (e.g., a hundred to thousand fold increase in total numbers of bacteria present) resulting in a polymicrobial flora. Studies point to changes in complex microbe-host interactions and individual variability in immune function as also being implicated in clinical manifestations of bacterial vaginosis.

While the infection involves a number of bacteria, it is believed that most infections start with Gardnerella vaginalis creating a biofilm on vaginal mucosa allowing other opportunistic bacteria to thrive. A definitive list of the bacterial organisms likely causing bacterial vaginosis is probably not possible due to variations in the observed dysbiosis on account of such factors as race, diet, age, and the like, nevertheless, representative organisms that are considered to be associated with bacterial vaginosis include, but are not limited to, one or more organisms from the following genera as well as the following specific organisms: Actinobacteria spp.; Anaerococcus spp.; Actinomyces spp. (e.g., Actinomyces naeslundii); Aggregatibacter spp. (e.g., Aggregatibacter actinomycetemcomitans); Atopobium spp. (e.g., Atopobium vaginae); Bacteroides spp. (e.g., Bacteroides ureolyticus); Bifidobacterium spp.; Clostridiales spp.; Collinsella spp. (e.g., Collinsella aerofaciens); Eggerthella spp. (e.g. E. lenta); Eubacterium spp.; Fusobacterium spp. (e.g., Fusobacterium nucleatum); Gardnerella spp. (e.g., Gardnerella vaginalis); Leptotrichia spp. (e.g., L. amnionii); Megasphaera spp.; Mobiluncus spp.; Mycoplasma spp. (e.g., M. hominis, and M. parvum); Peptococcus spp.; Peptoniphilus spp.; Peptostreptococcus spp. (e.g., P. anaerobius); Porphyromonas spp. (e.g., Porphyromonas gingivalis); Prevotella spp. (e.g., P. bivia, P. disiens, and P. intermedia); Slackia spp.; Sneathia spp. (e.g., Sneathia sanguinegens); Tannerella spp. (e.g., Tannerella forsythia); Treponema spp. (e.g., Treponema denticola); Ureaplasma spp. (e.g., Ureaplasma urealyticum); and Veillonella spp. (e.g., Veillonella parvula). Most species of bacteria believed to be associated with bacterial vaginosis appear to have proinflammatory characteristics.

### Diagnosis of Bacterial Vaginosis

Common symptoms of bacterial vaginosis include increased white or gray colored vaginal discharge that is often malodorous. Burning sensations during urination may occur. Itching is uncommon. Occasionally, there may be no symptoms. The onset of bacterial vaginosis approximately doubles the risk of infection by a number of other sexually transmitted infections, including HIV/AIDS. The infections also increases the risk of early delivery among pregnant women. Risk factors include frequent douching, new or multiple sex partners, use of antibiotics, and use of intrauterine devices, among others. The infection is not considered a sexually transmitted infection. Diagnosis is based on the observed symptoms, and may be verified by testing the vaginal discharge and finding a higher than normal vaginal pH, and large numbers of representative bacteria. Bacterial vaginosis is often confused with a vaginal yeast infection or infection with Trichomonas.

Diagnostic procedures by a qualified practitioner are typically required to detect whether an infection is present, and to determine the nature of the infection. Diagnostic procedures often include visual microscopy, usually of a vaginal wet mount sample, and culture of the vaginal discharge. The color, consistency, acidity/alkalinity, and other characteristics of the discharge may be predictive of the causative agent.

The diagnostic tests for bacterial vaginosis include the so-called Amsel criteria and the Nugent Gram-stain scoring system. Bacterial vaginosis diagnosis under the Amsel criteria requires that at least three of four of the following criteria are met for a given patient: 1) characteristic vaginal discharge; 2) vaginal pH>4.5; 3) positive amine test, meaning release of a malodorous primary amines (e.g., putrescine, cadaverine, and trimethylamine) from a vaginal sample on the addition of the KOH; and 4)>20% of the epithelial cells present in the wet mount slide of the vaginal discharge identified as “clue cells” (mucosal cells coated with Gram-variable coccobacilli consistent with the organism Gardnerella vaginalis). (Amsel R., et al., Am. J. Med., 74(1):14-22 (1983)). These primary amines can increase vaginal pH and promote the growth of other anaerobic bacteria implicated in bacterial vaginosis.

Another common test for bacterial vaginosis is the Nugent Gram-stain scoring system. This test involves assessment of a normally prepared Gram stain for relative abundance of three morphotypes of bacteria and calculation of the “Nugent score” based on decrease in Lactobacilli (scored as 0 to 4), abundance of small Gram-negative and variable rods, Bacteroides and Gardnerella morphotypes (scored as 0 to 4), and abundance of curved gram-variable rods, Mobiluncus spp., morphotype (scored as 0 to 2). Nugent scores can range from 0 to 10, with scores of 0-3 deemed normal (non-BV), 4-6 intermediate, and 7-10 (positive for BV). The Nugent scoring system is somewhat more objective than the Amsel criteria, and is regarded as a more reproducible and predictive means of diagnosing bacterial vaginosis. (See, Bennett, J., and Mandell D., Bennett's principles and practice of infectious diseases, Philadelphia, PA: Elsevier/Saunders (2015)).

Recently developed diagnostic for bacterial vaginosis make use of molecular biology techniques such as the use of a variety of DNA-based analysis tools, such as broad-range and quantitative PCR (e.g., AFFIRM® Microbial Identification Test, Beckton Dickinson, Franklin Lakes, NJ).

### pH Modulating Compositions, Formulations, Administration and Dosing Considerations

The practice of the present invention will employ, unless otherwise indicated, conventional techniques of preparing pharmaceutical formulations as well as drug delivery and dosing techniques which are well known in the art. Generally speaking, the compositions of the present invention may be prepared by means standard in the art. A number of standard text are known in the art regarding preparation and formulation considerations. (See e.g., Remington's Pharmaceutical Sciences).

In certain embodiments, the compositions of the present disclosure are sterile and, optionally, preservative-free. In other embodiments the compositions are sterile, optionally preservative-free, formulated in a single-use or unit-dose formats. In still further embodiments the sterile formulations contain one or more preservatives, stabilizers, sugars, or sugar alcohols.

The methods and compositions of the present invention provide treatments for maintaining healthy populations of vaginal flora (e.g., bacteria) and/or the health and integrity of mucosal surfaces (e.g., vaginal tissues, structures, and the like) in a subject in order to confer a medicinal or therapeutic benefit in the subject by the administration of an effective dose of the one or more of compositions described herein. Methods of administering the compounds of the invention may be by metered dose or by one or more controlled release devices. The compositions may be in unit dosage forms suitable for single administration of precise dosages.

In some embodiments, the concentration of one or more of the compounds provided in the pharmaceutical compositions of the present invention is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v or v/v.

In yet some other embodiments, the concentration of one or more of the compounds comprising the present invention is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In still some other embodiments, the concentration of one or more of the compounds comprising the present invention is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the concentration of one or more of the compounds comprising the present invention is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In some other embodiments, the amount of one or more of the compounds comprising the present invention is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of one or more of the compounds comprising the present invention is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

Other embodiments provide, amounts of one or more of the compounds of the present invention in the range of 0.0001-10 g, 0.0005-9 g, 0.001-8 g, 0.005-7 g, 0.01-6 g, 0.05-5 g, 0.1-4 g, 0.5-4 g, 1-3 g, or 1-10 g.

The target dose may be administered in a single dose. Alternatively, the target dose may be administered in about or more than about 1, 2, 3, 4, 5, 6, 7, 8, 9, 10, 11, 12, 13, 14, 15, 16, 17, 18, 19, 20, 25, 30, or more doses.

The administration schedule may be repeated according to any prescribed regimen, including any administration schedule described herein. The compositions of the present invention may be administered in one dose or multiple dosages. Methods of determining the most effective means and dosage of administration are well known to those of skill in the art and will vary with the particular compositions used, the purpose of the use, the target cells or tissues being contacted, and the subject being treated. Single or multiple administrations (e.g., about or more than about 1, 2, 3, 4, 5, 6, 7, 8, 9, or 10, or 50, more doses) over the course of from 1, 2, 3, 4, 5, 6, 7, 8, 9, or 10, or 50, or more, minutes, hours, days, weeks, months, or even years.

In some particularly preferred embodiments, one dose of the composition is administered (e.g., transvaginal administration) every 1-3, 1-7, 1-10, 1-12, 1-14, 1-28, or 1-30, or more, day(s) as prescribed by a physician or as otherwise deemed necessary for maintaining the desired vaginal pH. Administration can be carried out with the dose level and pattern being selected by the treating physician. It is known in the art that due to intersubject variability in compound pharmacokinetics, individualization of dosing regimens is necessary for optimal therapy. Dosing for compositions of the present invention may be found by routine experimentation in light of the instant disclosure and one's skill in the art.

Additionally, it is to be noted that, similar to the approaches described in the fields of medicinal and pharmaceutical chemistry, a suitable pharmaceutical preparation may also include, optionally, in addition to one or more compounds of the present invention, other agents, including, but not limited to, excipients, diluents, extenders, stabilizers, colors, flavors, formulating agents (e.g., gels and thickeners), antioxidants (e.g., ascorbic acid, butylated hydroxyanisole, butylated hydroxytoluene, citric acid, EDTA, phosphoric acid, sodium ascorbate, sodium metabisulfite, tartaric acid, tertiary butyl hydroquinone), preservatives, sterile aqueous solutions, buffers, sugars, and the like, as are generally known and accepted.

In other embodiments, one or more additional small molecule drug and/or biological agents may be preferentially combined with the one or more compounds of the present invention to achieve a beneficial, or even synergistic, outcome in the subject.

### Devices and Applicators

An applicator device suitable for instilling/depositing the compositions into the vagina is typically employed when the compositions are formulated as gel or gel like-products (e.g., barrel and plunger type devices). Applicator devices that find use in the administration of the various embodiments of the present invention include, but are not limited to, those described in U.S. 5,397,312; U.S. Pat. Nos. 5,860,946; 7,591,808; 9,517,328; and 9,884,173, and the like. The minimum requirements for suitable applicators is an adequate delivery or deposition of the desired amount (e.g., from about 100%, 99.99%, 99.98%, 99.97%, . . . to about 99%, 99.9%, 99.8%, 99.7%, . . . to about 98%, . . . to about 90%, . . . to about 80%, . . . to about 70%, . . . to about 60%, . . . to about 50%, . . . to about 40% . . . of the desired amount) of composition at the intended site(s) over the intended delivery time. Suitable multiuse dose and reusable applicators are similarly contemplated by the present invention and known in the art.

Accordingly, in certain preferred embodiments, suitable vaginal applicators may be used to administer the pharmaceutical formulations of the present invention to exterior and/or interior surfaces of the vagina and/or anorectal areas including, the perineum and rectum. The compositions may be delivered locally to the site of application or regionally to the surrounding area. In a particularly preferred embodiment, the formulations are delivered transvaginally to the region of the upper vaginal vault for the modulation of intravaginal pH levels. Alternatively, an applicator can be used for rectal administration of compositions such as to deliver the drug to the colon or the anal canal.

A physician can use standard tests to determine the efficacy of the various embodiments of the inventive compositions and methods in modulating vaginal pH levels. However, in addition to vaginal pH levels, the physician also may consider quality of life, comfort, and hygiene related issues in evaluating efficacy of a particular treatment regime and adjust specific administration schedules.

Certain compounds of the present invention are also useful as co-therapeutic compounds for use in combination with other one or more additional agents/drug substances, for example, but not limited to, agents such as antimicrobials, bactericides, bacteriostatic agents, antivirals, antifungal agents, anti-oncolytic agents, cancer drugs and therapies, and the like, that are typically administered into the vagina, or as these one or more additional agents/drug substances are used in the vaginal and in female reproductive healthcare.

It will be readily apparent to those skilled in the art that other suitable modifications and adaptations of the compositions and methods of the invention described herein are obvious and may be made using suitable equivalents without departing from the scope of the invention or the embodiments disclosed herein.

Having now described the present invention in detail, the same will be more clearly understood by reference to the following examples, which are included for purposes of illustration only and are not intended to limit the invention.

### Example 1

### Formulation of the pH Modulating Compositions

This Example describes methods and formulations for producing the compositions.

| Component | Amount (%) |
|---|---|
| Alginic acid USP | 4.25 |
| Xanthan gum | 3.00 |
| Glycerin | 10.00 |
| Lactic acid | 2.40 |
| Citric acid | 1.00 |
| Potassium acid | 0.40 |
| tartrate | |
| Benzoic acid | 0.20 |
| Sodium hydroxide | adjustable |
| Distilled water | 100.00 |

All chemical components and agents of the compositions are at least USP, USP-NF, or NF grade.

The pH of the composition is adjusted to a pH of about 3.5 to about 3.6 with sodium hydroxide. Compositions described in the Example are expected to demonstrate good stability and good vaginal pH modulation. The compositions in this Example as well as generally described and claimed herein can be formulated, tested, and used as described in U.S. Pat. No. 6,706,276.

The composition of this invention are generally prepared using conventional gel preparation techniques as known in the pharmaceutical and compounding sciences. Preferably the buffering agents are fully solubilized in the final product. It is also preferable that production methods employed are worked so as to minimize the entrapment of air in the gel as much as practicable.

This Example provides several methods by which the compositions of the invention can be made using laboratory scale equipment. Of course, other methods (i.e., different orders of addition of components as well as variation of other variables) can be used so and optimized for production on commercial scale while maintaining the desirable properties of the compositions described herein.

### Method 1

Using the formulation presented above, benzoic acid (4.0 g) is added to stirred water (950 ml) followed by the addition of sodium hydroxide (150 ml of a 1N solution or any other combination of volume and normality to provide equivalent amounts of sodium hydroxide). Potassium acid tartrate (8.0 g), citric acid mono hydrate (20.0 g), and lactic acid (40.0 ml) are added to the mixture; the pH adjusted to about 3.3 to about 3.6 with 1N sodium hydroxide (pH adjustment step 1). Alginic acid (85 g) is added to the stirred solution in small increments to insure a uniform dispersion and avoid air entrapment. Sodium hydroxide (230 ml of a 1N solution or any other combination volume and normality to provide equivalent amounts of sodium hydroxide) is then added to the solution and stirred for 10 minutes. The pH is measured and the pH adjusted to from about 3.3 to about 3.6 (using 1N sodium hydroxide if necessary; pH adjustment step 2). Stirring continues until a uniform mixture is obtained.

In a separate container, xanthan gum (60 g) is mixed with glycerin (160 ml) and stirred until uniform and then slowly mixed with the uniform mixture just described. An amount of water equal to 220 ml minus the volume of water used in pH adjustment steps 1 and 2 is then added to the mixture. The volume of added water will, of course, vary depending upon the strength of sodium hydroxide used in pH adjustment steps 1 and 2. The mixture is stirred for short period (about 15 min) and then allowed to stand for a short period of time (about 10 min). Stirring is then continued until a uniform gel consistency is obtained. The pH is checked and should be in the range of about 3.25 to about 3.80, and more preferably in the range of about from 3.37 and 3.52. If necessary, the pH is adjusted to bring it into the desired range by addition of sodium hydroxide solution.

### Method 2

Using the same formulation, benzoic acid (4.0 g) is dissolved in water (950 ml) with stirring (i.e., magnetic, mechanical, rotatory, vibratory, or ultrasonic stirrer and the like). Without waiting for complete dissolution of benzoic acid, sodium hydroxide (150 ml of a 1N solution or any other combination of volume and normality to provide equivalent amounts of sodium hydroxide) is added and stirring continued. Potassium acid tartrate (8.0 g) is then added to the mixture. The stirring speed or efficiency can be increased to ensure dissolution of the components. Citric acid monohydrate (20.0 g) is then added with stirring. Lactic acid (40.0 ml) is then transferred to the mixture (a small volume of distilled water is used to rinse the transfer vessel) and the pH adjusted to about 3.3 to about 3.6 with 1N sodium hydroxide (pH adjustment step 1). The buffer salts and acids can be dissolved or added in any order. Alginic acid (85 g) is added to the stirred solution in small increments (about one half to one spoonful per addition in batch laboratory scale equipment) to insure that the alginic acid is uniformly dispersed in the mixture without significant entrapment of air. Sodium hydroxide (230 ml of 1N solution or any other combination of volume and normality to provide equivalent amounts of sodium hydroxide) is then added to the solution and stirred for 10 minutes. The pH is measured and the pH adjusted to about 3.3 to about 3.6 (using 1N sodium hydroxide if necessary; pH adjustment step 2). Stirring continues for approximately 30 minutes or for a sufficient period of time to ensure uniform mixing.

In a separate container, xanthan gum (60 g) is mixed with glycerin (160 ml) and stirred until uniform. The uniform solution just prepared is then slowly transferred to the container with xanthan gum and glycerin with continuous stirring. The volumes of 1N sodium hydroxide used in the pH adjusting steps 1 and 2 are added and subtracted from 220 ml. An amount of water equal to 220 ml minus the volume of water used in pH adjustment steps 1 and 2 is then added to the mixture. The volume of added water will, of course, vary depending upon the strength of sodium hydroxide used pH adjustment steps 1 and 2. The mixture is stirred so as to obtain a reasonably uniform mixture without an excessive amount of air entrapment in the thickened preparation. The mixture is allowed to stand for about 10 minutes and then stirred for sufficient time to obtain a uniform gel consistency (normally about 15 minutes). The pH is checked and should be in the range of about 3.25 to about 3.80, and more preferably in the range of about from 3.37 and 3.52. If necessary, the pH can be adjusted to bring it into the desired range by addition of sodium hydroxide solution.

It is contemplated that compositions prepared using Method 1 or Method 2 (in this Example) will have essentially the same properties. For clinical use, the compositions should, of course, be analyzed to determine if product specifications for various physicochemical parameters (e.g., color, consistency, gel separation, odor, final pH (3.37-3.52), pH modulating capacity, and concentration of any added active ingredient) are achieved. The pH modulating capacity should be sufficient so that the pH of a mixture of 400 μl 1N NaOH in 40 ml of a 5 percent aqueous solution of the composition is not more than pH 4.55. Those skilled in the art will understand some product specifications or parameters (e.g., color or odor) are not expected to significantly impact clinical effectiveness.

In some embodiments, an additional active ingredient (i.e., antimicrobial and/or contraceptive agents) can be added at an appropriate stage in this Example, depending upon the physicochemical properties of the added ingredient. For example, a surfactant (e.g., Nonoxynol-9), which has a tendency to induce foaming, can be incorporated into the xanthan gum and glycerol mixture. Other antimicrobial and/or contraceptive agents can be incorporated at any time during the preparation of the gel and most preferably before the final consistency is attained so as to avoid entrapment of air in the gel.

### Example 2

### Study of Vaginal pH Modulation

This Example describes a scheme for conducting a randomized, placebo-controlled, double-blind study to determine the pH modulating characteristics of the composition.

Eligible subjects will include women aged 18 to 45 years who have regular menstrual cycles and agree to abstain from sexual intercourse, douching, or use of any intravaginal devices for at least 24 hours prior to study initiation. Exclusionary criteria will include symptoms of urinary tract infection, sexually transmitted infection, bacterial vaginosis, or yeast infection.

Subjects who are positive for any Amsel's criteria (i.e., clue cells, positive whiff test, discharge and/or elevated pH) can still be enrolled as long as they are asymptomatic at the entry visit. Subjects who are positive for one or more Amsel criteria will be provided the option for immediate treatment of their asymptomatic bacterial vaginosis with the potential for later entry into the study, or treatment at the end of the study. Subjects will be excluded from beginning the study based on engaging in sexual intercourse, douching, or use of any form of vaginal suppository or intravaginal device within 24 hours of study initiation. Subjects will also be excluded from beginning the study if they are pregnant or breastfeeding, or have symptoms of urinary tract infection, bacterial vaginosis, or yeast infection. Other exclusion criteria will include use of vaginal medications or suppositories, feminine sprays, genital wipes, or contraceptive spermicides; abnormal vaginal discharge within 48 hours of screening; menstruation or the expectation of menstruation during the 7-day study period; and use of vaginal contraceptives. All study participants will provide written informed consent prior to enrollment.

Following enrollment, an equal (or approximately equal) number of subjects are randomly assigned to one of five treatment groups in a 1:1:1:1:1 fashion to receive a single dose of composition at 5 g, 4 g, or 3 g, or placebo 4 g (e.g., an isotonic non-buffering gel containing 2.7% hydroxyethylcellulose, sodium chloride, sodium hydroxide, sorbic acid, and purified water adjusted to pH 4.5) or no treatment.

Randomization is accomplished using one or more known techniques. The randomization list is generated by a biostatistician and transferred to a designated data management administrator. The study biostatistician and all other study personnel will remain blinded until the study database is finalized and locked and instructions are provided for unblinding.

The investigational compositions are provided in blinded fashion. Composition containers are dispensed via lot number. An interactive web response system (IWRS) is utilized to provide the composition identification (e.g., lot numbers) for dispensing. Subject randomization numbers are available upon entry of subject eligibility information into the IWRS. Study treatments are provided in pre-filled, single-use applicators sealed with foil overwraps. A single dose of the composition is administered intravaginally by the clinician on Day 0 and subjects are double blinded to receive either composition 5 g, 4 g, or 3 g, or placebo 4 g, and will remain blinded for the duration of the study. All subjects are instructed to lay flat for at least 30 minutes following administration of the composition. Subjects in the no-treatment group are told that are receiving the composition.

Vaginal pH measurements are obtained from each subject prior to the first administration of the composition and at 1 hour and 6 hours post-administration (Day 0). Vaginal pH measurements will be taken from two different positions in the vagina to account for potential incomplete distribution of the composition or the placebo. Subjects will trained to self-collect vaginal swabs and to perform the vaginal pH measurements. At 12 hours post-treatment, subjects will self-collect vaginal swabs and conduct pH measurements recording their results for later review.

Subjects will stay overnight at a research domiciliary unit during which time an additional vaginal pH measurement will be taken prior to discharge at 24 hours post-administration of the composition. Clinicians will obtain adverse event (AE) assessment data while subjects are in the domiciliary unit at each vaginal pH measurement (at 1, 6, 12, and 24 hours post-administration). Subjects will be provided with appropriate pH testing supplies and a diary to record pH measurements when they are discharged. Subjects will collect vaginal swabs and perform pH measurements on Days 2-6 recording their pH values and any AE in the provided diary. At Day 7, participants will return to the testing center for their final study visit where staff will measure their vaginal pH and discuss any vaginal discomfort and AEs experienced during the study.

The primary efficacy endpoint will be the change in subject's vaginal pH from baseline during the study following administration of a single dose of the composition, placebo, or no treatment. The analysis population includes all subjects who received any amount of the composition as well as enrolled subjects who did not receive the composition (no-treatment group); analysis will be performed based on treatment received.

The statistical approach will be to assess differences in pH with descriptive statistical summaries to estimate the potential effect size provided by the treatments. Summaries for the vaginal pH data, including change from baseline, are provided for each evaluation time point by treatment group and overall for all active treatment groups combined. Comparisons across the treatment groups with respect to change from baseline in vaginal pH are performed using analyses of variance (ANOVA). Post-ANOVA pairwise comparisons of each study treatment versus the placebo treatment and versus no treatment are also assessed. Subject demographics (age, gender, race, and ethnicity) and physical characteristics (weight, height, and body mass index (BMI) at screening and AEs are summarized descriptively by treatment group and overall.

All AEs following enrollment are collected and recorded and are categorized according to the current Medical Dictionary for Regulatory Activities coding (System Organ Class and Preferred Term). Relationship to study treatment, action taken, duration of the event, and the outcome of the event are also summarized.

### INCORPORATION BY REFERENCE

The contents of all cited references including literature references as well as all foreign patents and patent foreign patent applications that are cited throughout this application are hereby expressly and specifically incorporated by reference in their entireties. Additionally, all U.S. Patent Publications, U.S. Patent Applications, and U.S. Patents are hereby expressly and specifically incorporated by reference in their entireties.

What is claimed is:

1. A method of modulating the pH level in the vagina, said method comprising administering an effective amount of pH modulating composition within the vagina, wherein the composition comprises:
1) about 1 to about 10 percent of a gelling agent selected from the group comprising alginic acid, chitosan, gellan gum, or poloxamer;
2) about 1 to about 10 percent of a mucoadhesive agent selected from the group comprising xanthan gum, hydroxypropyl cellulose, hydroxypropyl methyl cellulose, sodium carboxymethyl cellulose, chitosan, olycarbophil, or a crosslinked polyacrylic acid;
3) about 1 to about 10 percent of an acid component selected from the group comprising lactic acid, citric acid, potassium acid tartrate, benzoic acid, alginic acid, sorbic acid, fumaric acid, ascorbic acid, stearic acid, oleic acid, tartaric acid, edetic acid, or malic acid; and
4) water; further
wherein the composition is suitable for application within the vagina;
wherein the composition forms a gel on contact with vaginal mucus; further
wherein the composition maintains an acidic vaginal pH of less than about 4.5.

2. The method of claim 1, wherein said acidic vaginal pH is from about 3.5 to about 4.5.

3. The method of claim 1, wherein said acidic vaginal pH is maintained for 1-10 days following administration of said pH modulating compound.

4. The method of claim 3, wherein said acidic vaginal pH is maintained for 7 days following administration of said pH modulating compound.

5. The method of claim 1, wherein said gelling agent comprises alginic acid.

6. The method of claim 1, wherein said mucoadhesive agent comprises xanthan gum.

7. The method of claim 1, wherein said acid component comprises lactic acid and potassium acid tartrate.

8. The method of claim 1, wherein said pH modulating composition is formulated as a suppository.
